(19) United States
(12) Patent Application Publication
(10) Pub. No.: US 2004/0158286 A1
(43) Pub. Date: Aug. 12, 2004

(54) Hemostatic tissue clamp

(76) Inventors: Daniel Roux, Rouffiac Tolosan (FR); Valerio Valentini, Montreal (CA); Anthony Paolitto, St. Leonard (CA)

Correspondence Address: ANTHONY PAOLITTO, CORONEO, INC., 9250 Avenue du Parc, Suite 514, Montreal, QC, H2N 1Z2, CA

(21) Appl. No.: 10/775,077

(22) Filed: Feb. 11, 2004

Related U.S. Application Data
(60) Provisional application No. 60/446,523, filed on Feb. 12, 2003.

(51) Int. Cl. A61B 17/44

(52) U.S. Cl. 606/205

(57) ABSTRACT

A tissue clamp for clamping a target tissue site includes a first jaw member and a second jaw member being movable between an open configuration and a clamping configuration. In the open configuration, the first and second jaw members are in a substantially spaced relationship relative to each other for allowing insertion of at least a portion of the target tissue site therebetween. In the closed configuration, the first and second jaw members are in a substantially proximal relationship relative to each other for exerting a hemostatic pressure on the inserted portion of the target tissue site. In the clamping configuration, the first and second jaw members together defining a substantially endless tissue contacting surface for exerting a hemostatic pressure substantially encompassing the target tissue site. A jaw actuating means mechanically coupled to the first and second jaw members is provided for actuating the first and second jaw members between the open and clamping configurations.

This application claims the benefits of U.S. Provisional Patent Application Serial No. 60/446,523, filed on Feb. 12, 2003.

### FIELD OF THE INVENTION

The present invention relates to the general field of medical accessories and is particularly concerned with a hemostatic tissue clamp.

### BACKGROUND OF THE INVENTION

There exists a plurality of situations wherein it is desirable to reversably limit the flow of blood in certain target anatomical sites. For example, in numerous surgeries, it is often desirable to temporarily occlude a blood vessel. Conventional hemostatic clamps such as the Fogarty clamp, the De Bakay “Atraugrip”, the Bulldog clamp or Pott's and Satinsky's peripheral vascular clamps are used extensively for occluding vessels.

Although these conventional clamps have proven to be somewhat satisfactory in most instances wherein occlusion of a vessel is required, they typically present major drawbacks when used for hemostatically clamping other anatomical sites such as sections of organs or more broadly sections of tissues in general.

Various examples exist wherein it would be desirable to temporarily prevent the flow of blood in a tissue section other than a vessel. The following disclosure will use as an example of such situations the specific context of a partial nephrectomy, also called nephron-sparing surgery (NSS). It should however be understood that the present invention could be used in various other contexts, including various types of surgeries performed on various organs or tissues without departing from the scope of the present invention.

NSS in itself may prove to be suitable in a variety of contexts. For example, the curative management of renal cell carcinoma (RCC) remains surgical. Recent advances in preoperative staging, specifically modern imaging techniques, and improvements in surgical techniques have made partial nephrectomy an attractive alternative to radical nephrectomy in selected patients.

NSS is more clearly indicated for cases in which a radical nephrectomy would render the patient anephric with a subsequent immediate need for dialysis. Synchronous bilateral tumours, tumours in a solitary kidney, or the presence of a poorly functional contralateral renal unit are generally absolute indications for NSS. The latter scenario could result from the concomitant presence of unilateral RCC and a contralateral kidney with disease processes (eg, chronic pyelonephritis, renal arterial disease, calculus disease) or the presence of systemic diseases (eg, diabetes).

Partial nephrectomy may also be considered the treatment of choice for certain benign conditions and localized pathology of the kidney. It allows for optimal surgical treatment and, at the same time, obviates overtreatment and nephron loss when possible and necessary. Examples of possible relatively more benign indications include traumatic irreversible injury to a localized portion of the kidney and removal of a benign renal tumour such as an oncocytoma, angiomyolipoma, or multilocular cyst. Other indications include an obstructed atrophied segment of a duplicated kidney, calculous disease of a renal segment with impaired drainage, and, rarely, renovascular hypertension with identifiable noncorrectible branch renal artery disease.

When considering RCC, various criteria are used to assist in the evaluation of the pertinence of NSS. In addition to size, the location of the lesion in the kidney is an important criterion when considering NSS. Admittedly, centrally located tumours that are close to the hilum and adjacent to the collecting system are technically more difficult to remove than exophytic peripheral lesions.

The clinical utility of NSS for RCC is revealed when several factors are considered. First, RCC usually does not become symptomatic until late in its course. Lesions detected incidentally tend to be smaller and of lesser grade, and thus more amenable to conservative surgery. The value of NSS is realized further when one considers the unreliability of current imaging studies in distinguishing between malignant and benign tumours of the kidney.

Also, the natural history and malignant potential of small RCC is not well understood. Although observation could be a viable option in elderly patients with high comorbidities, NSS allows for curative surgery and elimination of uncertainty in the average patient with acceptable expected longevity. The goals of conservative resection of RCC are complete local surgical removal of the malignancy and preservation of adequate renal function. This is a delicate balance, which makes renal-preserving surgery, at times, both challenging and controversial.

Intraoperative renal ultrasound is increasingly being used during intrarenal surgery and has played a role in determining if patients are suitable for partial versus radical nephrectomy. Technical advances in the development of sonographic instrumentation have made this possible. These advances include the development of high-frequency multi-Hertz transducers offering a marked improvement in resolution, the development of miniature, intraoperative transducers that facilitate access into the surgical field, and the compactness of current model US machines that allow easy transport and mobility into the operating room suite. Also, the refinement of color and duplex Doppler sonography and the addition of power Doppler sonography have made intraoperative ultrasonography an integral component in the management of patients undergoing partial nephrectomy.

In patients undergoing partial nephrectomy, ultrasound can delineate a tumour in relation to the hilar anatomy and can demarcate the boundary of a surgical margin, thereby preserving the maximum amount of uninvolved parenchyma while still obtaining negative surgical margins. Color and power Doppler sonography can identify arteries, veins, and the urinary collecting system near the potential resection site, and the thickness of a renal parenchymal margin between tumour and vessel may be estimated.

Vessels around the tumour are delineated, which facilitates dissection, and the success of revascularization may be assessed using color Doppler sonography. The presence of tumour thrombus in the renal vein may be determined. Additionally, vascular structures (arteries and veins) may be differentiated from nonvascular structures such as cysts or a dilated calyx.

In addition to the standard imaging modalities, newer techniques have recently been proposed in an attempt to assist the surgeon in planning the best approach to remove the tumour. Helical CT combined with three-dimensional volume rendering has recently been shown to accurately depict both the renal parenchyma and the vascular anatomy, thus providing the surgeon with a three-dimensional depiction of the tumour in relation to the critical components of the kidney.

Several surgical techniques are available for performing nephron-sparing surgery in patients with renal tumours. The five main surgical processes include enucleation of tissue, polar segmental nephrectomy, wedge resection, major transverse resection, and extracorporeal partial nephrectomy followed by renal autotransplantation.

All of these techniques require steady vascular control and thorough hemostasis, avoidance of renal ischemia, complete tumour removal with free margins, and efficient closure of the intrarenal collecting system. Finally, an adequate postoperative renal function must be maintained since a functioning renal remnant of at least twenty percent (20%) of one kidney is necessary to avoid end-stage renal failure. However, it is important not to compromise the extent of the surgical procedure to preserve renal function at the expense of an incomplete resection.

Postoperative renal insufficiency typically results from a combination of intraoperative ischemia and loss of functioning renal parenchyma. The extent of renal insufficiency varies, and its degree is reflected by the increase of retention parameters such as creatinine, blood urea, and potassium. Severe renal insufficiency may require temporary dialysis. If the compensatory hypertrophy of the remnant kidney tissue cannot compensate for the loss of renal function, a permanent insufficiency requiring permanent dialysis may result.

The main steps of conventional partial nephrectomy include initiating diuresis with intravenous mannitol and a loop diuretic (eg, furosemid) intraoperatively, with generous hydration before any interruption in the renal circulation. Mannitol is infused before anticipated renal occlusion. This agent not only induces osmotic diurisis but also is a free radical scavenger that can minimize ischemic insult from arterial clamping and the ultimate risk of postoperative acute tubular necrosis.

An incision is performed either of the bilateral subcostal or thoracoabdominal type. Usually the subcostal incision is used. The thoracoabdominal incision is preferred when the tumour is large and at the upper pole of the kidney. After opening the abdomen, the colon is moved to expose the kidney.

The renal artery is temporarily clamped to reduce bleeding. Typically, the renal artery is occluded with an atraumatic vascular Bulldog clamp. The renal vein may remain non-occluded since retrograde profusion of the kidney might minimize the chance for acute tubular necrosis postoperatively.

The kidney is dissected from the surrounding tissue from outside the renal fascia.

The tumour is removed with a margin of normal tissue. The calyxes and renal pelvis that have been cut through are carefully closed with sutures. The cut end of the kidney is covered with fat, fascia or peritoneum. The clamp on the renal artery is removed and all bleeding is controlled prior to the incision being closed.

In situations wherein relatively sizeable lesions are resected, temporary arterial occlusion together with hypothermia may be required. Hence, when larger tumours are being resected, it may be preferable to apply iced saline and to allow the kidney to cool for adequate core renal hypothermia. It would thus be desirable to provide a tool that could simultaneously, or independently, act as a cooling means for providing adequate core renal hypothermia.

Preoperative definition of the renal vasculature is more imperative if a larger partial resection is contemplated. When in doubt, the appropriate segmental artery supplying the tumour can be identified by injection of indigo-carmine. It is usually recommended that excessive dissection be avoided and that surrounding perivascular adventitial layers be left intact to serve as cushions if the application of a vascular clamp is contemplated. This reduces the risk of intimal damage to the artery, which can result in arterial thrombosis.

Once the tumour has been removed, typically, the edges of the parenchymal defect are approximated and the defect is closed using suitable material. When large polar resections are approached, they usually require ligation of the segmental arteries and veins supplying the tumour and the corresponding section of the kidney.

One of the main drawbacks associated with the conventional partial nephrectomy method is that clamping of the renal artery causes ischemia of the whole kidney. Although the ischemia is typically transient it may nevertheless lead to renal insufficiency if the arterial clamp time is extended. Attention to intraoperative measures to decrease the possibility of this complication, such as hydrating preoperatively, correcting electrolyte abnormalities, using mannitol and potentially using surface hypothermia may prove to be insufficient in some unfortunate instances. Some unfortunate patients may hence need renal replacement therapy, for example hemodialysis.

As is well known, ischemia is a condition of tissue anoxia due to a stoppage of the inflow of arterial blood to body tissue. Reperfusion is the restoration of blood flow to the tissue previously rendered ischemic.

The technical literature reflects a significant effort in the medical research community directed to the development of an understanding of the damage observed in reperfused ischemic tissue. In fact, researchers have found that significant tissue damage resulting after a period of tissue ischemia, followed by reperfusion, occurs not only during the period of circulatory arrest, but during the period of reperfusion. Indeed, a relatively large portion of the total injuries seen after five to sixty minute periods of circulatory arrest may actually develop during the reperfusion stage. Such tissue damage is known as reperfusion injury.

Many medical researchers have proposed that the tissue damage associated with the so-called reperfusion injury is due to the abnormally high concentration of a species identified as a superoxide anion which is rapidly produced in previously hypoxic tissue upon the restoration of oxygenated blood flow to the hypoxic tissue. Thus, while oxygen is necessary to restore normal metabolism in hypoxic tissue, body chemistry during the period of hypoxia changes to favour the production of tissue damaging superoxide anions at a rate far above the rate such anions are produced during normal metabolism, and far above the rate that the body's own protective chemistry can handle.

Clamping and subsequent release of the renal artery may hence potentially lead not only to ischemia injury but also to reperfusion injuries. Some authorities believe that irreversible renal lesions occur when total renal ischemia resulting from clamping of the renal artery exceeds twenty minutes.

Also, typically, during the conventional partial nephrectomy, the parenchyma is malleable due to the arterial occlusion. However, when the renal tissue instead of the renal arteria is being squeezed for a hemostasis, the parenchyma may be less malleable and, hence, it is desirable to provide a tissue clamping tool that will exert sufficient pressure to facilitate the operative steps.

Another troublesome and potentially relatively more common intraoperative complication of the conventional partial nephrectomy method is excessive bleeding. In this respect, meticulous dissection, attention to detail and ligation of intraparenchymal vessels are of paramount importance. Easy access to the renal hilum, provided by early identification and isolation of the renal artery, provides additional safety of prompt arterial occlusion when excessive bleeding precludes a clear surgical field and adequate visualization. However, in some situations, this may prove to be insufficient potentially leading to the need for embolization or re-exploration in the case of severe intractable bleeding.

In an attempt to circumvent the hereinabove mentioned disadvantages associated with clamping of the renal artery during conventional nephron sparing or partial nephrectomy, some surgeons have attempted to clamp a segment of tissue surrounding the mass to be excised hence limiting the ischemia to the tissue about to be removed and its immediate periphery. Although reducing ischemia to the remainder of the kidney is theoretically appealing, attempts at clamping tumour-adjacent kidney tissue instead of the renal artery during partial nephrectomy have proven to be unsuccessful.

Problems associated with attempts at clamping kidney tissue instead of the kidney arteria may be, at least partially imputable to the use of conventional vascular clamps to perform the tissue clamping operation. As is well known, conventional vascular clamps typically include a pair of pivoting arms with a clamping jaw rigidly attached to a distal end of each pivoting arm.

The clamping jaws are movable between an open configuration wherein they allow insertion of a vessel therebetween, and a closed configuration wherein they allow the application of a clamping force on the vessel. Clamping typically results in complete vascular occlusion.

The process of clamping generates loci of high pressure far in excess of the pressure in the blood vessel itself. Conventional clamps such as the Fogarty clamp, the De Bakay “Atraugrip”, the Bulldog clamp or Pott's and Satinsky's peripheral vascular clamps exert relatively high pressures, in some cases up to nine bars on clamped blood vessels.

One of the drawbacks associated with conventional vascular clamps when used for clamping tissue, is that the applied pressure is distributed in a non-uniform manner at the interface between the clamping jaw and the tissue. Indeed, the conventional clamping jaws typically being of the scissor type create a gradient of applied pressure along the clamping jaws with the higher pressure being located adjacent to the proximal end located towards the hinge.

This leads to excessively high pressures in some areas potentially leading to undue injury of adjacent tissue and to insufficient pressure at distal locations leading to unsuitable hemostasis. In view of the fact that systemic blood pressure is at least one order of magnitude lower than pressure applied to the tissue by conventional clamps, it becomes evident that suitable hemostasis could be achieved at far lower pressures than those exerted adjacent to the proximal end of the jaws.

Furthermore, the configuration of most conventional vascular clamps has further proven to be unsuitable since it prevents insertion of body tissues of various configurations in size. It would hence be desirable to provide a clamping tool allowing for the tissue to be surrounded by a uniform external pressure field.

Also, conventional vascular clamp are not well suited for minimizing hemorrhage through the use of hypothermia. Furthermore, at least some of them lack features precluding their use in the context of endoscopic surgery, vacuum assisted surgery and the like. Accordingly, there exists a need for a hemostatic tissue clamp.

### SUMMARY OF THE INVENTION

In accordance with the present invention, there is provided a hemostatic tissue clamp for clamping a target tissue site, the tissue clamp comprising: a first jaw member and a second jaw member, the first and second jaw members being movable between an open configuration and a clamping configuration wherein when the first and second jaw members are in the open configuration the first and second jaw members are in a substantially spaced relationship relative to each other for allowing insertion of at least a portion of the target tissue site therebetween, and wherein when the jaw members are in the clamping configuration the first and second jaw are in a substantially proximal relationship relative to each other for exerting a hemostatic pressure on the portion of the target tissue site; the first and second jaw members together defining a substantially endless tissue contacting surface for exerting a hemostatic pressure substantially encompassing the target tissue site when in the clamping configuration; a jaw actuating means mechanically coupled to the first and second jaw members for actuating the first and second jaw members between the open and clamping configurations.

Advantages of the present invention include that the proposed hemostatic tissue clamp allows for the hemostasis to be induced relatively proximally to the organ target site instead of requiring that a larger organ segment including a relatively large healthy section be subjected to ischemia such as when an artery is clamped. For example, in the case of a partial nephrectomy, the use of a hemostatic tissue clamp in accordance with the present invention obviates the need for clamping the renal artery and, hence, for the need to subject healthy nephrons to potentially damaging ischemia. The prevention of potentially damaging ischemia to healthy nephrons, in turn, may reduce the risks of renal failure with its associated humanly and monetarily costly hemodialysis.

Also, the proposed tissue clamping tool, by obviating the need for extensive surgical dissection of the vasculature, may potentially substantially reduce the duration of given surgeries.

Furthermore, the proposed hemostatic tissue clamping implement is designed so as to provide an efficient hemostatic action through a set of quick and ergonomic steps.

Still furthermore, in at least some embodiments of the present invention, the proposed hemostatic tissue clamp is designed so as to increase the surgical field or improve surgeon access thereto by being, at least in part, displaceable relative thereto.

Also, in at least some embodiments of the present invention, the proposed hemostatic tissue clamp is designed so as to reduce the potential trauma to the clamped tissue section imputable to the pressures exerted thereon.

In yet at least some other embodiments of the present invention, the proposed hemostatic tissue clamp is provided with sealing means for at least partially sealing part of the surgical field so as to reduce the risk of disseminating tumorous tissue cells.

Also, in at least some embodiments of the present invention, the proposed hemostatic tissue clamp is designed so as to provide suction on the target organ so as to facilitate the isolation of a tumour located within.

Still furthermore, in yet at least some other embodiments of the present invention, the proposed hemostatic tissue clamp is provided with means for attachment thereof to various types of surgical platforms.

Also, in at least some embodiments of the present invention, the proposed hemostatic tissue clamp is provided with cooling means for selectively inducing hypothermia to target anatomic sites.

In yet at least some other embodiments of the present invention, the proposed hemostatic tissue clamp is designed so as to be configurable and sizeable so as to be customizable to accommodate various tumour sizes and configurations in various locations.

Also, the proposed hemostatic tissue clamp, in at least some embodiments thereof, is designed so as to be usable in an endoscopic approach and, hence, is designed so as to be insertable within a conventional trocar.

Still furthermore, the proposed hemostatic tissue clamp is designed so as to be manufacturable using conventional forms of manufacturing in order to provide a hemostatic tissue clamp economically feasible, long-lasting and relatively trouble-free in operation.

### BRIEF DESCRIPTION OF THE DRAWINGS

Various embodiments of the present invention will now be disclosed, by way of example, in reference to the following drawings in which:

FIG. 1 in a perspective view illustrates a hemostatic tissue clamp in accordance with an embodiment of the present invention being used for hemostatically clamping a section of a kidney, prior to removal of a tumorous lesion therefrom;

FIG. 2 in a perspective view with sections taken out illustrates a hemostatic tissue clamp in accordance with a second embodiment of the present invention being used for hemostatically clamping a tissue section of a kidney, the clamp being shown with arm segments thereof in a folded configuration so as to minimize obstruction of the surgical field;

FIG. 3 in a partial perspective view with sections taken out illustrates a hemostatic tissue clamp in accordance with a third embodiment of the present invention, the clamp being shown hemostatically clamping a tissue segment of a kidney and in a folded configuration wherein the surgical site is substantially unobstructed thereby;

FIG. 4 in a partial perspective view with sections taken out illustrates a hemostatic tissue clamp in accordance with a fourth embodiment of the present invention, the tissue clamp being shown in a clamping configuration;

FIG. 5a in a top view illustrates a hemostatic tissue clamp in accordance with a fifth embodiment of the present invention, the hemostatic tissue clamp being shown in an open configuration;

FIG. 5d in a side elevational view illustrates the hemostatic tissue clamp shown in FIG. 5a;

FIG. 5e in a partial cross-sectional view taken along arrows 5e-5e of FIG. 5d illustrates part of the hemostatic tissue clamp shown in FIGS. 5a and 5d;

FIGS. 5b, 5f, 5g and 5h in partial front views taken along arrows 5b-5b of FIG. 5a illustrates the surface texture of at least one of the jaws of the hemostatic tissue clamp shown in FIG. 5a;

FIGS. 5c, 5i, 5j, 5k and 5l in transversal cross-sectional views taken along arrows 5c-5c of FIG. 5a illustrate the cross-sectional configurations of at least one of the jaws of the hemostatic tissue clamp shown in FIG. 5a;

FIG. 5m in an elevational front view ilustrates the configuration of an alternative embodiment of a jaw member part of a hemostatic tissue clamp in accordance with the present invention;

FIG. 5n in an elevational front view ilustrates the configuration of yet another alternative embodiment of a jaw member part of a hemostatic tissue clamp in accordance with the present invention;

FIG. 6a in a partial perspective view with sections taken out illustrates a hemostatic tissue clamp in accordance with a sixth embodiment of the present invention, the hemostatic tissue clamp being shown in a clamping configuration and having a sealing membrane mounted thereon;

FIG. 6b in a partial cross-sectional view taken along arrows 6b-6b of FIG. 6a illustrates the cross-sectional configuration of the clamp and membrane shown in FIG. 6a as the clamp is being used for clamping a section of a kidney;

FIG. 7a in a partial perspective view with sections taken out illustrates a hemostatic tissue clamp in accordance with a seventh embodiment of the present invention, the hemostatic tissue clamp being shown with a suction skirt and hose mounted thereon in an open configuration;

FIG. 7b in a partial perspective view with sections taken out illustrates the hemostatic tissue clamp as shown in FIG. 7a in a suctioning enclosed configuration;

FIG. 7c in a partial perspective view with sections taken out illustrates the hemostatic tissue clamp shown in FIGS. 7a and 7b in a closed configuration with the suction skirt removed therefrom;

FIG. 8a illustrates a hemostatic tissue clamp in accordance with an eighth embodiment of the present invention, the tissue clamp being shown attached to a surgical platform including a tissue retracting means;

FIG. 8d in a partial perspective view with sections taken out illustrates a hemostatic tissue clamp in accordance with a ninth embodiment of the present invention, the tissue clamp being shown attached to a surgical retractor;

FIG. 9a in a partial perspective view with sections taken out illustrates a hemostatic tissue clamp in accordance with a tenth embodiment of the present invention, the tissue clamp being shown with tissue cooling means mounted thereon;

FIG. 9b in a partial perspective view with sections taken out illustrates the hemostatic tissue clamp in accordance with an eleventh embodiment of the present invention, the tissue clamp being shown with an alternative tissue cooling means mounted thereon;

FIG. 10a in a partial perspective view with sections taken out illustrates a hemostatic tissue clamp in accordance with a twelfth embodiment of the present invention, the tissue clamp being shown partially inserted through a conventional trocar during a laborascopic surgery;

FIG. 10b in a partial perspective view with sections taken out illustrates part of the tissue clamp shown in FIG. 10a, the tissue clamp being shown in an elongated and retracted configuration allowing insertion thereof within the lumen of the trocar;

FIG. 10c in a partial perspective view with sections taken out illustrates the hemostatic tissue clamp shown in FIGS. 10a and 10b in a closed configuration about to be deployed;

FIG. 10d in a partial perspective view with sections taken out illustrates the tissue clamp as shown in FIGS. 10a through 10c in a clamping configuration wherein it is being used for clamping part of a tumorous kidney.

FIGS. 11a to 11d illustrate a hemostatic tissue clamp according to the present invention, provided with an energy transmission means able to contact clamped body tissue and transfer energy to or from it.

### DETAILED DESCRIPTION

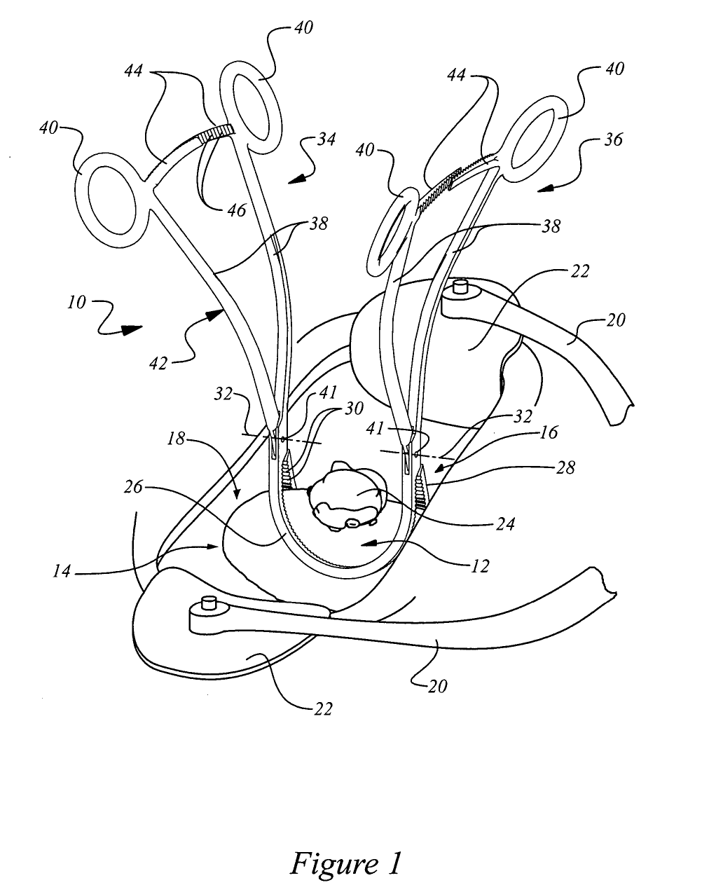

Referring to FIG. 1, there is shown a hemostatic tissue clamp 10 in accordance with an embodiment of the present invention. The tissue clamp 10 is shown clamping a target section 12 of an organ 14 part of a surgical field 16. FIG. 1 also illustrates an organ access aperture 18 maintained in an open configuration by a pair of retractor arms 20 and associated retractor blades or plates 22.

The tissue clamp 10 is shown throughout the figures as being used in the context of a nephron-sparing or partial nephrectomy for removing a generally substantially ovaloid-shaped mass 24 from an externally located basilar segment of a kidney. It should however be understood that the tissue clamp 10 could be used in numerous other contexts such as for removing other types of anatomical components or subcomponents having other configurations and in other locations of human or animal bodies or for providing selective ischemia in totally different contexts without departing from the scope of the present invention.

The tissue clamp 10 includes at least two jaw segments 26, 28 displaceable relative to each other between an open configuration wherein they are in a substantially spaced relationship relative to each other, and a closed configuration wherein the segments 26, 28 are in a generally proximate configuration.

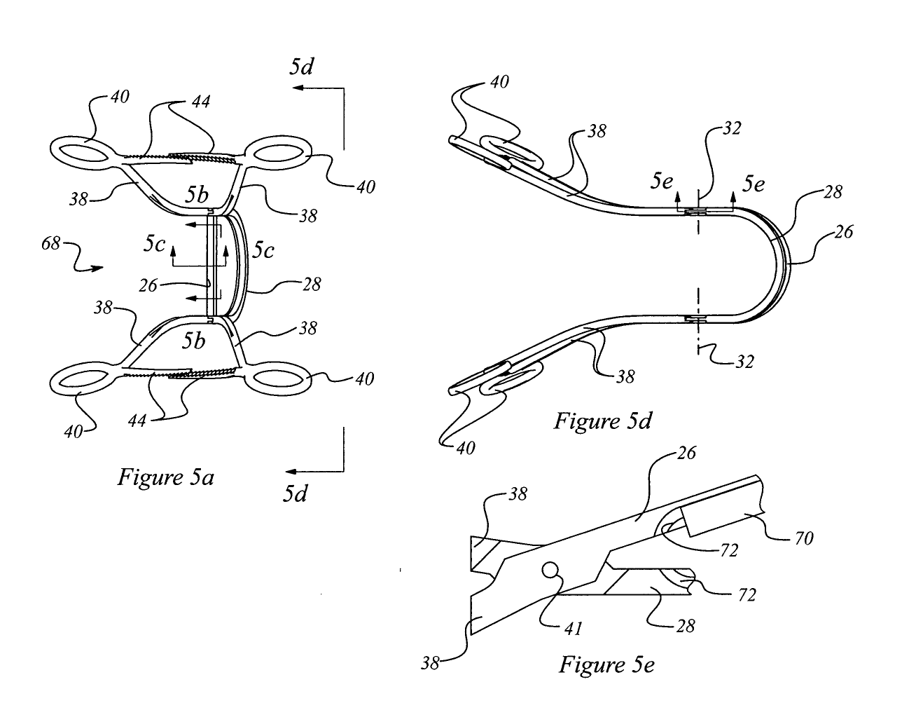

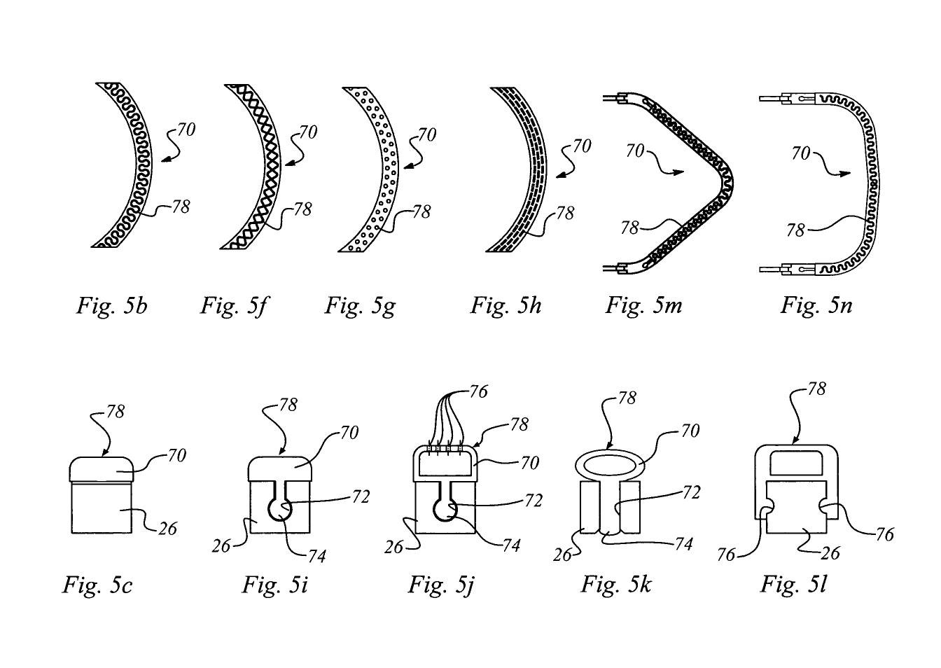

The jaw segments 26, 28 are configured and sized for providing a hemostatic clamping action at the peripheral border of the target tissue section 12 when in the closed configuration. In the embodiments shown throughout most of the figures, both jaw segments 26, 28 have a generally arcuate and U-shaped configuration. It should however be understood that the jaw segments 26, 28 could have any other suitable configuration including the configurations shown in FIG. 5. For example, FIGS. 5m and 5n show jaw members having respectively a generally <<V>>-shaped configuration and a generally flatenned <<U>>-shaped configuration.

Also, although the jaw segments 26, 28 are shown throughout the figures as having generally similar configurations relative to each other, it should be understood that the jaw segments 26, 28 could have different configurations as long as they define segments thereof cooperating for providing a clamping action when in the closed configuration.

In the embodiments shown throughout most of the figures, the jaw segments 26, 28 each define corresponding tissue contacting surfaces 30 for contacting the peripheral border of the target tissue segment 12. In the embodiment shown throughout most of the figures, the tissue contacting surfaces 30 have a generally flat configuration and are provided with serrations formed thereon. It should however be understood that the tissue contacting surfaces 30 could assume other configurations such as a generally V-shaped grooved configuration, a continuous or discrete or segmented configuration or any other suitable configuration without departing from the scope of the present invention. Also, the tissue contacting surfaces 30 could be provided with other types of friction enhancing textures or other characteristics without departing from the scope of the present invention. Such textures include an array of ridges, grooves, raised pedestals, raised truncated pyramids, depressed dimples, striations, or other like features. Alternatively, the tissue contacting surfaces can be coated or covered with a hydrogel type layer well suited for enhancing friction with or adherence to contacted tissue.

Preferably, the jaw segments 26, 28 are configured and sized so that the tissue contacting surfaces 30 thereof together form a generally endless loop. In other words, the jaw segments 26, 28 are preferably configured and sized so as to form a substantially closed perimeter, substantially in register with the peripheral border of the target tissue segment 12. The target tissue segment 12 is hence generally encompassed so as to provide a substantially efficient hemostatic action.

Preferably, the jaw segments 26, 28 are pivotally linked together adjacent both ends thereof. Alternatively, the jaw segments 26, 28 could be pivotally linked together adjacent to a single end thereof, or otherwise moveably connected together so as to be able to move between the jaw opened and closed configurations. In situations such as shown throughout most of the figures wherein the jaw segments 26, 28 are pivotally attached together, they are typically configured and sized so as to pivot at both ends thereof about co-linear pivotal axes 32.

Alternatively, in embodiments of the invention (not shown) more than two jaw members, or segments, could be used for forming a substantially closed perimeter clamp. In such instances, the jaw segments may be pivotal or otherwise moveable between the closed and opened configurations. The jaw segments may also be positioned and sized so that their movement towards the closed configuration is synchronized according to a predetermined closing pattern so as to bring about a predetermined clamping action taking into consideration the specificities of the tissue being clamped such as its specific vascular pattern. For example, the clamping action may be modulated so as to assist in evacuating arterial or venous blood therefrom prior to the target tissue segment being hemostatically segregated from the remainder of the body.

The tissue clamp 10 is also provided with actuating means for moving the jaw segments 26, 28 between the open and clamping, or closed, configurations. In the embodiment shown in FIG. 1, the actuating means includes a first and a second pair 34, 36 of actuating arms 38. The actuating arms 38 are mechanically coupled to the clamping segments 26, 28 to allow pivotal action thereof between the opened and closed configurations. Typically, the actuating arms 38 extend integrally into corresponding jaw segments 26, 28 and are pivotally attached together by a hinge pin 41 for movement in a scissor-like fashion. Alternatively, the actuating arms 38 could be releasable or otherwise attached to the clamping segments 26, 28. Typically, although by no means exclusively, the actuating arms 38 may be provided with conventional finger loops 40 extending therefrom at a distal end thereof for allowing insertion thereinto of fingers of the intended user. Also, typically, although by no means exclusively, the actuating arms 38 may be bent longitudinally and outwardly about an arm elbow section 42 so as to substantially diverge away from the surgical field 16 in a direction leading away from the latter.

The tissue clamp 10 is optionally further provided with a clamp locking means extending therefrom for locking the jaw members 26, 28 in a predetermined spacing relationship relative to each other. In the embodiment shown in FIG. 1, the clamp locking means includes locking tongs 44 extending from the actuating arms 38 adjacent the finger loops 40. The locking tongs 44 are provided with cooperating ratchet teeth 46 extending therefrom for releasably locking the actuating arms 38 and, hence, the jaw members 26, 28 in a predetermined spatial relationship relative to each other.

In short, in the embodiment shown in FIG. 1, the tissue clamp 10 may be approximated to a pair of vascular clamps mounted in an opposed relationship relative to each other and having their corresponding opposed clamping jaws attached together. The use of two pairs 34, 36 of actuating arms 38 allows for a generally evenly distributed actuating force on the jaw members 26, 28 so as to provide a generally evenly distributed hemostatic pressure on the peripheral border of the target tissue 12.

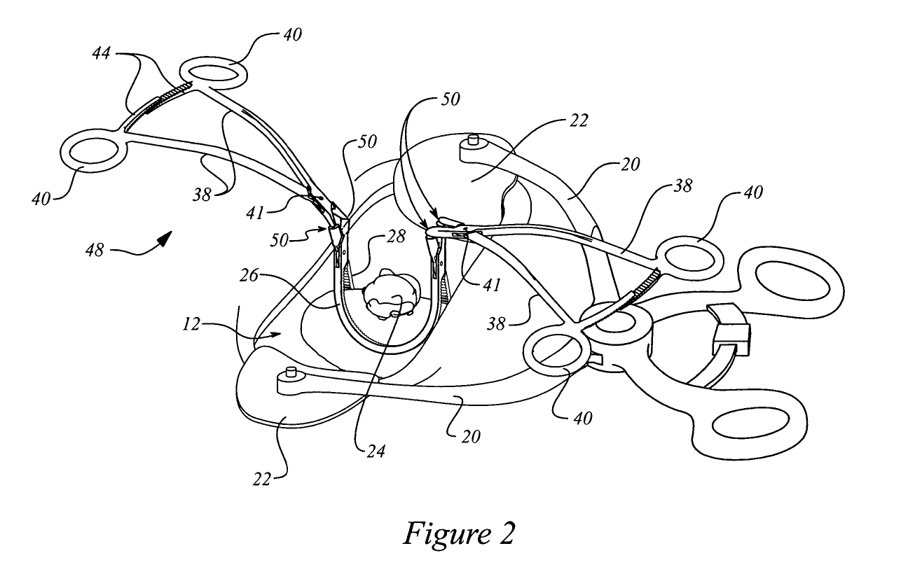

Referring now more specifically to FIG. 2, there is shown a hemostatic tissue clamp 48 in accordance with a second embodiment of the present invention, the hemostatic tissue clamp 48 is substantially similar to the hemostatic tissue clamp 10 and, hence, similar reference numerals will be used to denote similar components.

One of the main differences between the embodiments 10 and 48 resides in that at least one and preferably both pairs 34, 36 of actuating arms 38 are collapsible or foldable so as to reduce obstruction thereby of the surgical field 16. In the embodiment shown in FIG. 2, both pairs 34, 36 of actuating arms 38 are folded outwardly in a direction generally parallel to the longitudinal axis of the organ 12, or outwardly away from tumor or mass 24. It should however be understood that the actuating arms 38 could be foldable or otherwise displaceable in any of one or more directions and through any suitable range of motion without departing from the scope of the present invention.

In the embodiment shown in FIG. 2, each actuating arm 38 is divided into a corresponding pair of arm segments mechanically coupled together by a ball joint-type of mechanism 50. The ball joint-type of mechanism 50 is located substantially adjacent to the hinge pin 41. It should however be understood that any other suitable movement allowing means could be provided including one or more segment movement allowing means positioned along each or only some of the actuating arms 38 at any locations therealong without departing from the scope of the present invention.

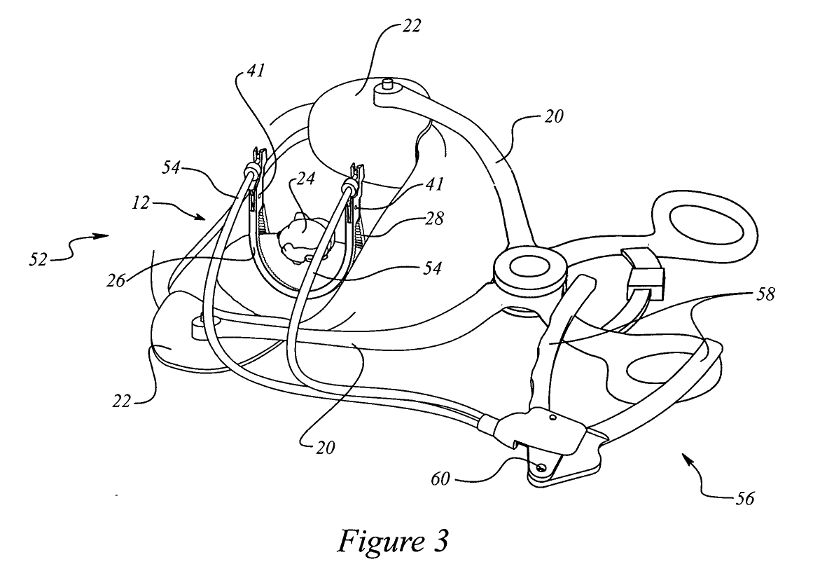

Referring now more specifically to FIG. 3, there is shown a hemostatic tissue clamp 52 in accordance with a third embodiment of the present invention. The hemostatic tissue clamp 52 is substantially similar to the hemostatic tissue clamp 10 and, hence, similar reference numerals will be used to denote similar components.

One of the main differences between the embodiment 52 and the embodiment 10 resides in the type of actuating means being used. In the embodiment 52, the actuating means includes at least one and preferably two actuating cables 54 mechanically coupled at a proximal end thereof to the jaw members 26, 28 and at a distal end thereof to hand-cable interface. Typically, the hand-cable interface includes a squeeze-type handle 56 defining a pair of handle levers 58 pivotally attached together about a lever hinge 60. Pivotal movement of the handle levers 58 is mechanically transmitted through the cable 54 into a corresponding pivotal movement of the jaw members 26, 28. Such configuration of hand-cable interface advantageously also provides force amplification; that is, the force applied at the handle levers 58 is augmented in magnitude to result in a force transmitted at cable 54 by virtue of lever hinge 60.

Typically, a push-pull type of cable slideably inserted within a corresponding sheath or sleeve may be used. The transmission cables 54 being preferably flexible may be typically positioned so as to free access to the surgical field 16.

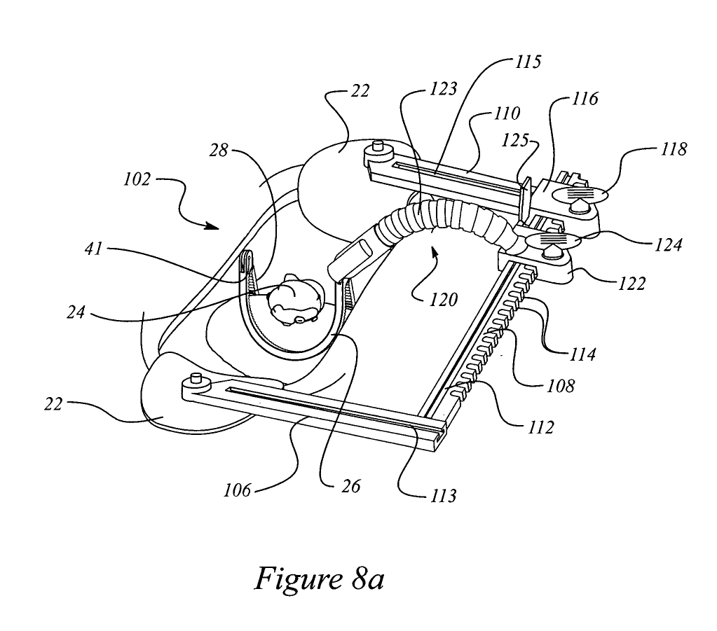

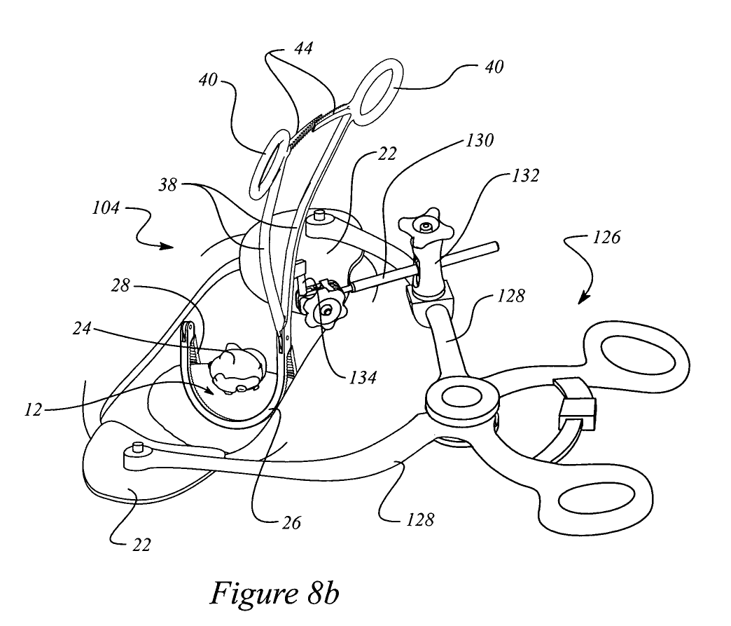

Alternatively, the sheath may be replaced by a plurality of pivotally-engaged, articulating sockets having substantially spherical mating ends (eg, such as sockets 123 shown in FIG. 8a), through which a transmission cable may slide. Such articulating socket arm can assume a multitude of configurations when the cable is not tensioned within the said sockets (i.e. jaws 26, 28 are in an open configuration). When the the transmission cable is tensioned by squeezing handle levers 58, in order to transmit the hemostatic clamping load on the jaws 26, 28, the articulating sockets become locked in their respective positions relative to each other, thereby assuming a fixed spatial relationship. As such, a desired arm configuration can be obtained that keeps surgical field 16 free from obstruction thereof.

Optionally, a handle locking mechanism (not shown) may be provided for selectively locking the handle levers 58 and, hence, the jaw segments 26, 28 in a predetermined spacing relationship relative to each other.

The use of handle levers 58 allows a palm grip using both the palm and the fingers of an intended user to exert a clamping force on the jaw members 26, 28. Hence, a greater force may be applied than with the use of the conventional finger loops 40 or eyelets shown in FIG. 1.

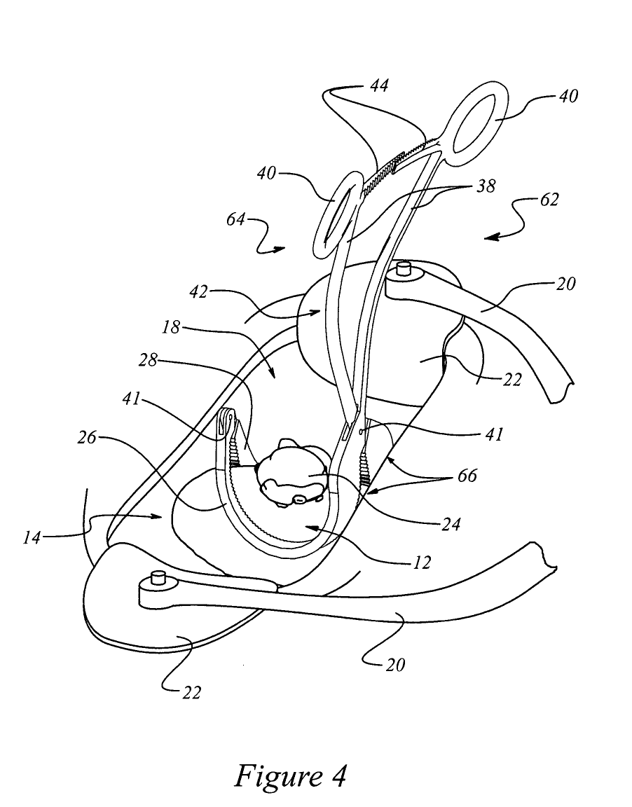

Referring to FIG. 4, there is shown a hemostatic tissue clamp 62 in accordance with a fourth embodiment of the present invention. The hemostatic tissue clamp 62 is similar to the hemostatic tissue clamp 10 and, hence, similar reference numerals will be used to denote similar components. Two of the main differences between embodiments 62 and 10 reside in the type of actuating means being used and the configuration of the jaw members 26, 28. The actuating means includes a single pair 64 of actuating arms 38.

In order to distribute the hemostatic pressure evenly on the peripheral border of the target tissue 12 and prevent distortion of the jaw members 26, 28, the latter are preferably designed so as to be structurally stiffer proximally to the actuating arms 38. Should the jaw member 26, 28 have a uniform stiffness therealong, they would have a tendency to distort more proximally than distally leading to uneven closure thereof in their closed configuration as they compress the peripheral border of the target tissue 12.

In the embodiment shown in FIG. 4, greater proximal stiffness is imputable to a larger cross-sectional area of the jaw members 26, 28 in their respective proximal region 66. However, numerous other methods could be used for obtaining higher stiffness in the proximal region 66 such as using a different material, providing a different curing for a metallic alloy, providing a stiffening sleeve or other stiffening geometries, or any other suitable means without departing from the scope of the present invention.

Referring now more specifically to FIGS. 5a through 5l, there is shown a hemostatic tissue clamp 68 in accordance with an embodiment of the present invention. The hemostatic tissue clamp 68 is substantially similar to the hemostatic tissue clamp 10 and, hence, similar reference numerals will be used to denote similar components.

One of the main differences between the embodiments 68 and 10 resides in the presence of at least one jaw sleeve 70 covering at least a portion of at least one and preferably both jaw members 26, 28.

The jaw sleeves 70 are preferably releasably mounted to the jaw members 26, 28. The jaw sleeves 70 may be releasably mounted to the jaw members 26, 28 through various sleeve-to-jaw releasable attachment means. As shown more specifically in FIG. 5e, one possible sleeve-to-jaw releasable attachment means includes at least one sleeve mounting or receiving keyway or channel 72 formed in at least a section of at least one of the jaw members 26, 28 for slideably receiving a corresponding attachment section of a jaw sleeve 70. As shown in FIGS. 5i through 5l, the sleeve receiving channel 72 may take any suitable form for receiving a substantially correspondingly shaped sleeve attachment protrusion, fitting or tongue 74.

As shown in FIGS. 5b, 5f, 5g and 5h, the sleeve 70 may be provided with various types of surface textures. It should be understood that the jaw sleeve 70 could be releasably attached to the jaw members 26, 28 using any other type of tongue and groove configuration or any other releasable fastening means without departing from the scope of the present invention. Sleeves may be designed with desired material properties for atraumatic, or less traumatic, clamping of tissue, thereby aiming to preserve healthy tissue while achieving hemostatic clamping proximal to target anatomic site 12. Material properties may also be selected to improve the adherence between the clamped tissue and jaw sleeve, thereby limiting the amount of slippage experienced as clamp jaws are moved from their open to their clamped configuration.

FIGS. 5b through 5l illustrate various jaw sleeve characteristics. For example, as shown in FIGS. 5c and 5i, the jaw sleeve 70 can be integrally filled with material or, alternatively, as shown in FIGS. 5j through 5l, the jaw sleeve 70 could be hollowed out and provided with a channel extending therealong. Also, as shown in FIG. 5j, the jaw sleeve 70 could be provided with jaw sleeve apertures 76 extending therethrough for acting as suction ports allowing a suctioning force to be transmitted to the tissue therethrough.

Also, the tissue contacting surface 78 of the jaw sleeve 70 could be provided with friction enhancing means or cushioning means without departing from the scope of the present invention. FIGS. 5b and 5f through 5h illustrate various types of relief configuration formed on the jaw tissue-contacting surface 78. Again, it should be understood that the patterns shown in FIGS. 5b and 5f through 5h are only shown by way of example and that other pattern configurations could be used without departing from the scope of the present invention. For example, the pattern on the jaw tissue-contacting surface 78 could be formed from protrusions extending therefrom, indentations formed therein or a combination of the latter.

The jaw sleeves 70 are typically formed out of a suitable polymeric and/or elastomeric resin approved for surgical use. The jaw sleeve 70 could be made out of an integral piece of material or, alternatively, could be formed out of a combination of materials or an anisotropic material without departing from the scope of the present invention. Alternatively, the tissue contacting surface 78 can be coated or covered with a hydrogel type layer well suited for enhancing friction with or adherence to contacted tissue.

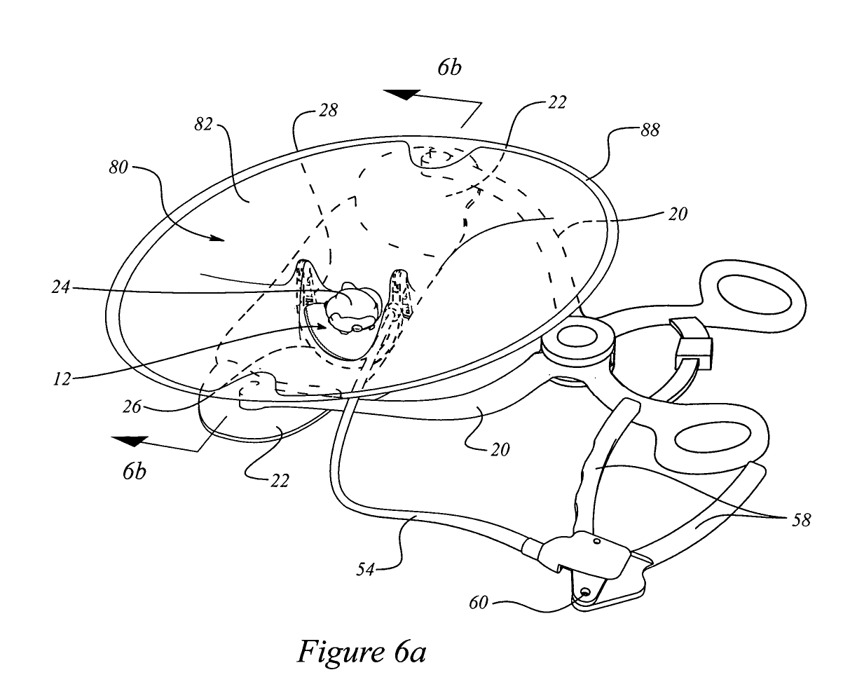

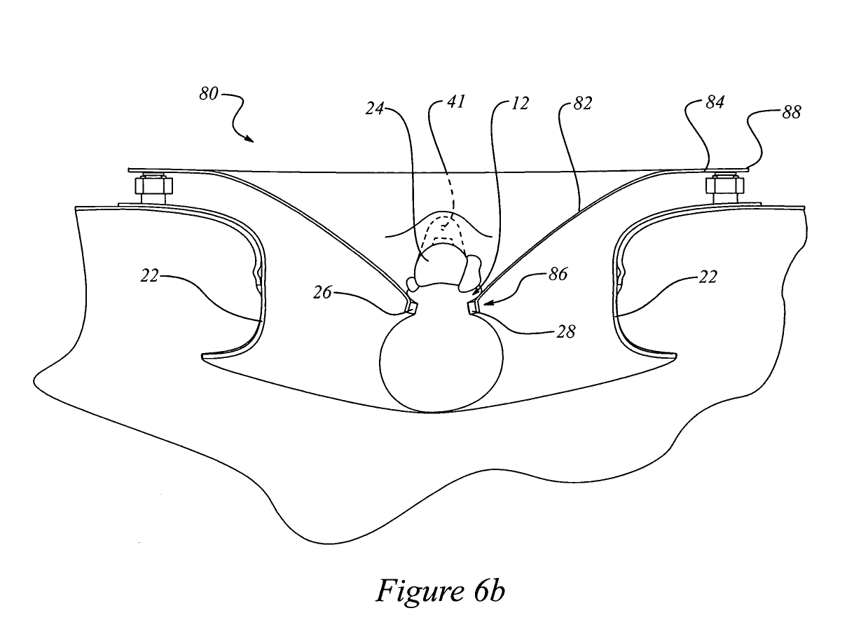

Referring now more specifically to FIGS. 6a and 6b, there is shown an hemostatic tissue clamp 80 in accordance with a 6^{th} embodiment of the present invention. The embodiment 80 is substantially similar to the embodiment 10 or 52 and, hence, similar reference numerals will be used to denote similar components.

One of the main differences between the embodiment 80 and the embodiment 10, 52 resides in the presence of a dissemination-preventing means for preventing or at least reducing the risk of disseminating potentially cancerous cells from the surgical field 16 to other parts of the body. The dissemination preventing means typically includes a shielding membrane 82. The shielding membrane is preferably made from polymeric or elastomeric material approved for surgical use.

The shielding membrane 82 typically defines a membrane outer peripheral edge 84 and a membrane inner peripheral edge 86 (FIG. 6b). The membrane inner peripheral edge 86 is typically releasably attached to the tissue clamp 80 using suitable releasable fastening means. Typically, although by no means exclusively, the membrane inner peripheral edge 86 is attached to the jaw members 26, 28. The membrane inner peripheral edge 86 may be either attached to the tissue clamp 80 prior to deploying the tissue clamp while the tissue clamp 80 is in its open configuration, or after tissue clamp 80 is deployed in its clamping configuration, with body tissue clamped within jaws 26, 28.

The shielding membrane 82 is typically deployed outwardly from the jaw members 26, 28 to an exteriorly positioned membrane attachment rim 88. The membrane attachment rim 88 is, in turn, typically mounted on a structure such as the retractor plates 22 (as shown), or alternatively, it may be attached to another location on a surgical platform or retractor, such as on arm 20 thereof. The membrane rim 88 may be fixedly or releasably attached to the retractor plates 22 and the membrane outer peripheral edge 84 may be permanently or releasably attached to the membrane rim 88. Also, it should be understood that, although the membrane 82 is shown as having a generally funnel-shaped configuration, the generally rounded membrane outer peripheral edge 84, the membrane 82 could assume other configurations without departing from the scope of the present invention.

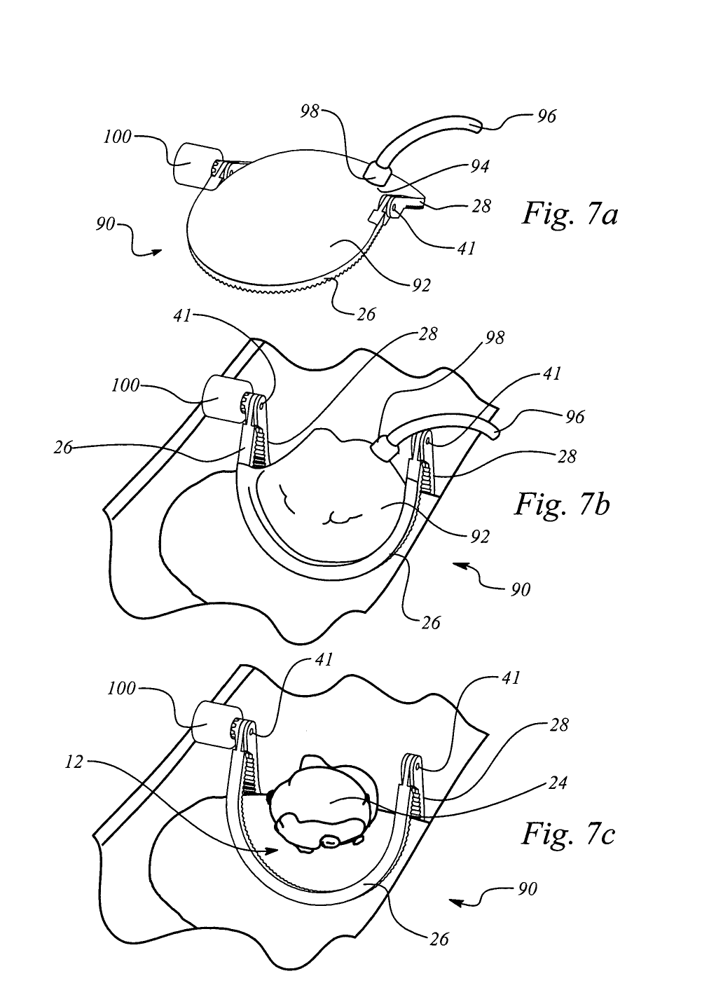

Referring now more specifically to FIGS. 7a through 7c, there is shown a hemostatic tissue clamp 90 in accordance with a seventh embodiment of the present invention. The tissue clamp 90 is substantially similar to the tissue clamp 10 and, hence, similar reference numerals will be used to denote similar components.

One of the main differences between the tissue clamp 90 and the tissue clamp 10 resides in the presence of a suction-providing means for allowing suctioning of the target anatomical site 12. The suction-providing means may take any suitable form. In the embodiment shown in FIGS. 7a through 7c the suction providing means includes a suctioning skirt 92 extending between the jaw members 26, 28 for performing a substantially air-tight and flexible pneumatic barrier therebetween. The suctioning skirt 92 is typically mounted on an exterior surface of the jaw members 26, 28 located opposite the tissue-contacting surface 30. Any type of suitable attachment means, preferably of the releasable type may be used for attaching the peripheral edge of the suctioning skirt 92 to the outer surface of the jaw members 26, 28.

The suctioning skirt 92 is provided with at least one suction aperture 94 extending therethrough. The suction aperture 94 allows pneumatic coupling thereto of a suction hose 96, to part of a suction-providing device (not shown). The suctioning skirt 92 may be provided with a pneumatic coupling 98 optionally having one-way or other types of valves formed therein for allowing coupling of the suction hose 96 thereto.

The suction-providing means may be used for many purposes. For example, the suction-providing means may be used for pneumatically biasing a more inwardly located tumorous mass towards a more superficially-positioned location for facilitating the clamping of a target section 12, and the removal of said tumorous mass thereof. FIG. 7b illustrates a situation wherein the jaw members 26, 28 are in their closed configuration and the suction providing means is deforming both the suction skirt 92 and the target anatomical zone 12 as an inwardly-located mass is systematically biased towards a more superficial location. FIG. 7c illustrates the jaws 26, 28 remaining in their closed configuration while the suctioning skirt 92 has been removed therefrom, in order to allow surgical access to the mass 24.

FIGS. 7a through 7c also illustrate yet another alternative embodiment of the actuating means. The actuating means is schematically illustrated as a generally cylindrical driving component 100 mechanically coupled to the jaw members 26, 28. The driving component 100 may be of any suitable type such as a pneumatic, hydraulic or electrical motor mechanically coupled to the jaw members 26, 28 by suitable coupling means such as a direct drive, a gear box or the like. A ratcheting mechanism may also be incorporated with the actuating means 100, thereby acting to maintain the clamping load at jaws 26, 28 when the actuating force at driving component 100 is released.

The driving component 100 may be actuated through any suitable actuating means such as pedal controls (not shown) allowing the surgeon or an assistant thereof to move the jaw members 26, 28 between their closed and open configuration without having to use their hands. The driving component 100 may also be voice actuated or otherwise selectively allowed to move the jaw components 26, 28 between closed configurations without departing from the scope of the present invention. Furthermore, it should be understood that the driving component 100 could be used with any of the embodiments shown throughout the Figures without departing from the scope of the present invention.

Referring now more specifically to FIGS. 8a and 8b, there is shown hemostatic tissue clamps 102 and 104 in accordance respectively with an 8^{th} and a 9^{th} embodiment of the present invention. The embodiments 102 and 104 are substantially similar to the embodiment 10 and, hence, similar reference numerals will be used to denote similar components.

One of the main differences between the embodiments 102, 104 and the embodiment 10 resides in the presence of a mounting means for mounting the jaw components 26, 28 to a structural component part of the surgical platform, thereby setting said jaws in a desired spatial relationship relative to said surgical platform. This advantageously allows the target anatomic site or zone 12 to be positioned and oriented within the surgical field, in a manner that improves surgical access to mass 24, and fixed in said position or orientation at least for part of the surgical intervention.

In the embodiment 102 shown in FIG. 8a, the retractor plates 22 are selectively maintained in a predetermined spaced relationship relative to each other by a rack-and-pinion type of structure including a fixed retractor arm 106 fixedly mounted to a rack bar 108 adjacent a first longitudinal end thereof, and a movable retractor arm 110 movably mounted on the rack bar 108 for slidable movement therealong.

The rack bar 108 is provided with a longitudinal guiding slot 112 and a set of rack teeth 114 extending therefrom. A cursor-type component 116 is mounted on the cursor or rack bar for incrementally adjustable movement therealong using typically a pinion type mechanism operable using a pinion handle 118.

The jaw members 26, 28 are attached on an adjustable mounting arm 120 adjacent the distal end thereof. The proximal end of the adjustable arm 120 is, in turn, attached to a cursor-type component 122 similar to the cursor component 116 having a pinion mechanism actuatable through a pinion handle 124 similar to the handle pinion 118. The adjustable arm 120 is typically, although by no means exclusively, of the segmented type allowing telescopic and bending adjustment thereof. For example, arm 120 may consist, at least in part, of a plurality of pivotally-engaged, articulating sockets 123 having substantially spherical mating ends. A transmission cable (not shown) passing through said arm 120, and through sockets 123, is mechanically coupled at the distal end to the jaws 26, 28 and at the proximal end to lever 125. Actuating lever 125 serves to move jaws 26, 28 from their open to their clamping configuration. Lever 125 may also be designed to simultaneously rigidify arm 120 in a manner that locks the relative position of the sockets 123 relative to one another. Alternatively, lever 125 may be designed to impart a tensioning load on a second tensioning cable which serves to rigidity said arm 120. The second tensioning cable may be in a co-axial relationship to the first transmission cable. Arm 120 may also be positioned in any location along longitudinal slot 112, or even in arm slots 113 or 115, in order to most optimally access the target site 12.

It should be understood that the embodiment shown in FIG. 8a is illustrated and described by way of example only and that other types of structures could be used without departing from the scope of the present invention. For example, other types of surgical platforms including other types of retractors could be used and other types of linking arms 120 could be used without departing from the scope of the present invention. For example, the embodiment 104 shown in FIG. 8b is mounted to a surgical platform including the scissor-type retracting structure 126 including a pair of retractor arms 128 pivotally attached together opposite the retractor plates 22.

The actuating arms 38 are attached to a mounting arm 130 adjacent the distal end thereof. Mounting arm 130 is slideably mounted to an arm-mounting structure 132, in turn, mounted on one of the retractor arms 128. The mounting arm 130 is slideably, pivotingly (i.e. able to pivot inwardly toward organ access aperture 18, and outwardly away from), and rotatingly (arm 130 is able to rotate about its centerline axis) attached to the arm-mounting component 132, and is provided with a tiltable end segment 134 for allowing adjustment of the position of the actuating arms 38. Again, it should be understood that the embodiment shown in FIG. 8b constitutes an example of numerous other types of embodiments illustrating the general concept of having a hemostatic tissue clamp provided with a means for attachment thereof to a surgical platform of any suitable type.

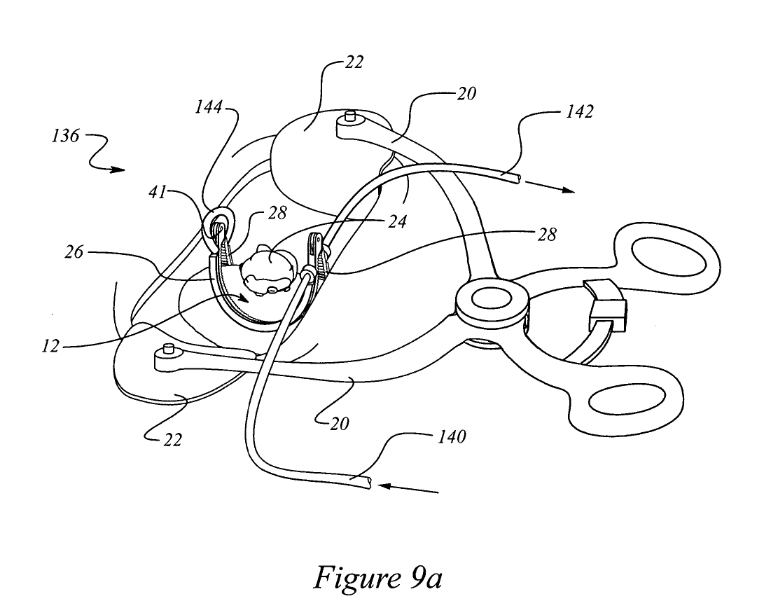

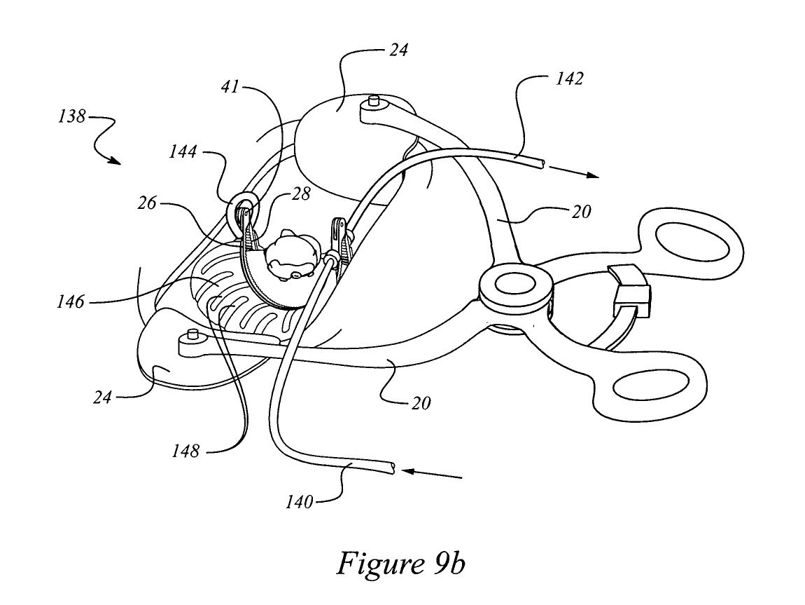

Referring to FIGS. 9a and 9b, there is shown hemostatic tissue clamps 136, 138 in accordance respectively with a 10^{th} and an 11^{th} embodiment of the present invention. The embodiments 136, 138 are substantially similar to the embodiment 10 and, hence, similar reference numerals will be used to denote similar components.

One of the main differences between the embodiments 136, 138 and the embodiment 10 resides in the presence of the cooling means for cooling at least part of the target anatomical site 12. In the embodiment 136 shown in FIG. 9a, the cooling means includes a cooling fluid inlet duct 140 and a cooling fluid outlet duct 142, both fluidly coupled to a fluid channel (not shown) extending at least partially through at least one and preferably both of the jaw components 26, 28. Typically, the linking duct 144 extends between the jaw components 26, 28 generally opposite the inlet and outlet fluid ducts 140, 142 for fluidly coupling the jaw components 26, 28 together.

A suitable cooling fluid is typically pumped by suitable pumping means through the fluid channel of the jaw components 26, 28 allowing for conductive cooling of the target anatomical site 12. The jaw components 26, 28 may also be optionally provided with temperature sensing means (not shown) for sensing the temperature of the target anatomical site.

One of the main differences between the embodiment 138 and the embodiment 136 resides in the presence of a cooling skirt 146 fluidly coupled to the fluid channels of the jaw components 26, 28. The cooling skirt 146 is provided with skirts channels 148 in fluid communication therebetween and with the fluid channels of the jaw components 26, 28. The skirt channels 148 are disposed according to a predetermined pattern allowing for a predetermined pattern of cooling the target anatomical site 12, or healthy portion of body organ at large. Typically, although by no means exclusively, the skirts channels 148 form a substantially serpentine-like configuration. Alternatively, the fluid channels in jaws 26, 28 may be on a separate fluid network than the skirt channels 148 in skirt 146.

Optionally, the cooling skirt 146 may be used for cooling an area adjacent the target anatomical site while the fluid circulating through the fluid channels of the jaw components 26, 28 may be at a sufficiently low temperature to produce necrosis of the tissue in contact therewith so as to facilitate severing thereof.

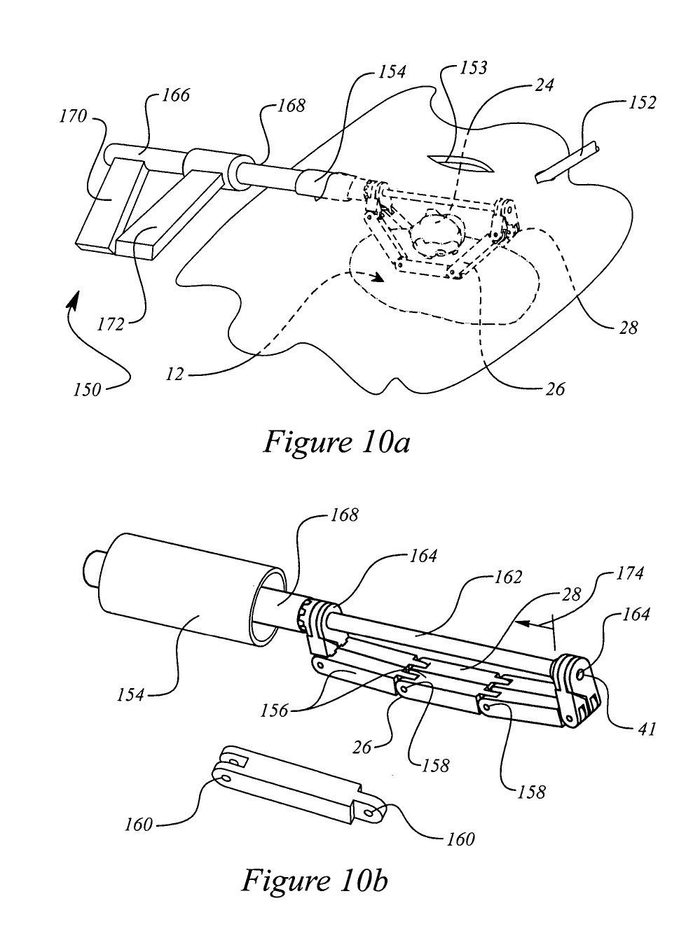

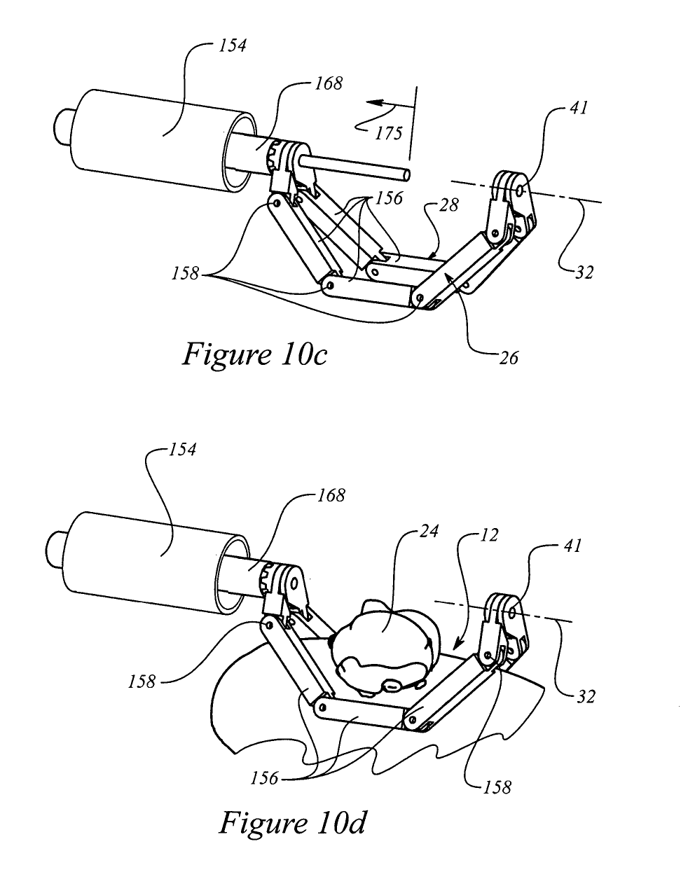

Referring now more specifically to FIGS. 10a through 10d, there is shown a hemostatic tissue clamp 150 in accordance with the 13^{th} embodiment of the present invention. The embodiment 150 is substantially similar to the embodiment 10 and, hence, similar reference numerals will be used to denote similar components.

One of the main differences between the embodiment 150 and embodiment 10 resides in that the embodiment 150 is specifically designed so as to be usable in the context of an endoscopic surgical procedure. As is well known, such laparoscopic surgical procedures are typically performed by initially inserting an inflating needle into the abdomen and injecting carbon dioxide or other suitable gases through the inflation needle into the peritonium to create a distended pneumoperitonium. Typically, although by no means exclusively, the peritonium is insufflated to a pressure substantially in the range of 14 to 18 mm of Hg.

Once the distended pneumoperitonium has been established, a primary trocar such as the trocar 152 is inserted into the peritonium through a small peri-umbilical incision or puncture site. Additional tubular trocars such as trocar 154 are then inserted into the peritonium at other sites of the abdominal mid-line or lateral to the midline.

Each trocar, such as trocars 152, 154 inserted into the abdomen is typically provided with a sealing or valving apparatus. Such sealing or valving apparatus operates to substantially prevent leakage from the pneumoperitonium when the trocar is inserted into the pneumoperitonium.

As is well known in the art, a puncturing stylet having a sharp tip is initially inserted through the lumen of the trocar for penetrating the peritonial membrane. Once the stylet has been withdrawn and removed, the tubular trocar may then be utilized as an access route or passageway for inserting and removing various surgical instruments, scopes, cannulae and/or other apparatus into the peritonial cavity. For instance, yet another trocar may be placed incision or puncture site 153 and serve as a passageway for surgical instrument used to incise tumorous mass 24. Alternatively, such surgical instrument may also be inserted in said incision 153 without the use of a trocar, at least for part of the surgical procedure.

In the embodiment shown in FIGS. 10a through 10d, the surgical tool is a hemostatic tissue clamp 150. The jaw components 26, 28 are typically formed out of substantially rectilinear jaw segments or links 56 pivotally attached together adjacent longitudinal ends thereof by suitable hinge means such as hinge pins 158 extending through corresponding hinge pin apertures 160.

A guide rod 162 extending through corresponding guide rod eyelets 164 positioned adjacent the longitudinal ends of the jaw components 26, 28 is used for maintaining the jaw components 26, 28 in a generally rectilinear configuration, hence preventing pivotal movement between the jaw links 156 against the action of gravity. Alternatively, a bias means such as a spring member (not shown) may be placed to react between two adjacent links to cause such links to pivot relative to one another and thereby assume a predetermined shape when guide rod 162 is withdrawn, and said pivotal movement is allowed.

The jaw components 26, 28 are mechanically coupled respectively to a first and a second transmission rod 166, 168 having corresponding handles 170, 172 extending therefrom for allowing selective pivotal movement of the jaw components 26, 28.

In use, the jaw components 26, 28, when in their rectilinear configuration shown in FIG. 10b are insertable into the pneumoperitonium such as shown in FIG. 10a through the trocar 154. Once inserted into the pneumoperitonium, the guide rod 162 is retracted according to arrow 174 allowing the jaw segments 156 to pivot relative to each other under the action of gravity. Optionally a biasing means such as a leaf-type spring or other types of suitable springs could be used for biasing the jaw segments to pivot relative to each other. Once the jaw components 26, 28 assume a generally rectilinear configuration such as shown in FIG. 10c, the jaw components 26, 28 may be rotated extra-corporeally using the handle components 170, 172 and corresponding transmission rod 166,168. Once the anatomic taget site 12 is clamped by jaws 26, 28, guide rod 162 may be decoupled from guide rod eyelet 164, and further retracted within transmission rod assembly 166,168 according to arrow 175, in a direction away from target anatomic site 12 (FIG. 10c). As such, free access to tumorous mass 24 is achieved.

It should be understood that numerous other structures and concepts can be used for providing hemostatic tissue clamps adapted for endoscopic use without departing from the scope of the present invention.

Also, it should be understood that the hemostatic tissue clamp, in accordance with the present invention could be provided with cryotherapy and/or radio frequency ablation means without departing from the scope of the present invention. The cryotherapy modality could be performed using a percutaneous approach using MRI and/or CT guidance.

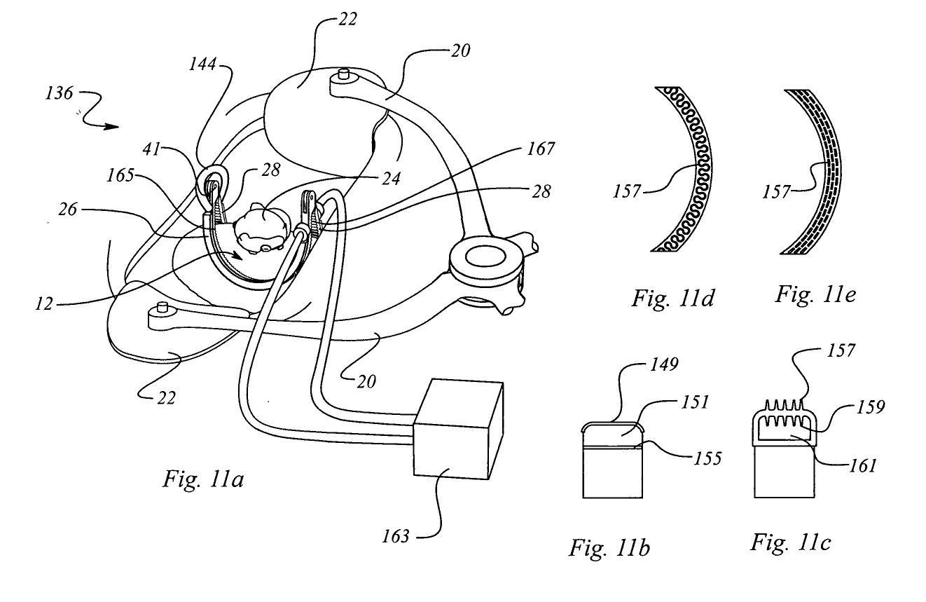

The hemostatic tissue clamp 139 illustrated in FIG. 11a is provided with an energy transmission means 165 and 167 that may be connected to any, or a combination of, a variety of sources 163. Such sources 163 may include, but are not limited to, a bipolar Radio-Frequency energy source, a microwave energy source, a cryogenic fluid source, and an ultrasonic energy source. The energy transmission means 165 and 167 of the tissue clamp 139 are preferably at least partially embedded within jaws 26 and 28, respectively. Alternatively, they may also be placed above, below, or atop of said jaws, but always respect an operational distance away from, or in contact with, body tissue when they are to be deployed. In use, when transmission means 165 and/or 167 are connected to an electrical source they may either heat the tissue in contact with the jaws 26, 28, as in the case where the transmission means in a heating element, or cool the tissue as in the case where the transmission means exploit the Thermoelectric Seebeck effect.

FIG. 11b illustrates a possible configuration wherein a thin foil 149 is applied to a non-conductive substrate 151 by any of a variety of means, such as adhesive bonding or electroplating, or other like viable means. The foil 149 then acts as one of two poles in the bipolar radio frequency source. Alternatively, said thin foil 149 can be a thin sheet of piezoelectric crystal bonded to conductive substrate 151 and can be excited via source 163. The conductive substrate 151 is bonded via insulating adhesive 155 to the hemostatic tissue clamp 139, and more specifically a jaw portion 26, 28 thereof. Alternatively, the thin foil 151 can be one side of any two members that form a thermoelectric series that will exploit the Seebeck effect when an electrical current is applied.

FIG. 11c illustrates a thermal energy transfer means that uses a fluid circulating within chamber 161 to either heat or cool the tissue in contact with jaws 26, 28. The fluid may be of any of a variety of sources, including but not limited to, liquefied gases such as nitrogen, supercooled solutions such as saline or glycol solutions, or heated fluids such as air, nitrogen, water, glycol solutions. Both the exterior and the interior of the fluid circulating chamber may be equipped with heat transfer augmentation fins 157 and 159 to improve the efficiency of the heat transfer with the contacted tissue.

FIGS. 11d and 11e show possible patterns or arrays for the energy transmission means 165, 167 located on the faces of jaws 26, 28.

1. A hemostatic tissue clamp for clamping a target tissue site, said tissue clamp comprising:
a first jaw member and a second jaw member, said first and second jaw members being movable between an open configuration and a clamping configuration, wherein when said first and second jaw members are in said open configuration said first and second jaw members are in a substantially spaced relationship relative to each other for allowing insertion of at least a portion of said target tissue site therebetween, and wherein when said jaw members are in said clamping configuration said first and second jaw are in a substantially proximal relationship relative to each other for exerting a hemostatic pressure on said portion of said target tissue site;
said first and second jaw members together defining a substantially endless tissue contacting surface for exerting a hemostatic pressure substantially encompassing said target tissue site when in said clamping configuration;
a jaw actuating means mechanically coupled to said first and second jaw members for actuating said first and second jaw members between said open and clamping configurations.
